(19) United States
(12) Patent Application Publication
(10) Pub. No.: US 2020/0406052 A1
(43) Pub. Date: Dec. 31, 2020

(54) Digestive Tract Capsule Endoscopy Integrated With Photodynamic Diagnosis And Therapy

(71) Applicant: Chinese Academy of Medical Sciences &Peking Union Medical College Institute of Biomedical Engineerin, Tianjin (CN)

(72) Inventors: Huijuan Yin, Tianjin (CN); Xiaoxi Dong, Tianjin (CN); Hongxiao Li, Tianjin (CN); He Huang, Tianjin (CN); Jian Zhang, Tianjin (CN); Yingxin Li, Tianjin (CN)

(73) Assignee: Chinese Academy of Medical Sciences &Peking Union Medical College Institute of Biomedical Engineerin, Tianjin (CN)

(21) Appl. No.: 16/734,000

(22) Filed: Jan. 3, 2020

(30) Foreign Application Priority Data
Jun. 25, 2019 (CN) 201910552076.8

(51) Int. Cl. A61N 5/06; A61B 1/04; A61B 1/00; A61B 1/06; A61B 1/273

(52) U.S. Cl. CPC A61N 5/0603; A61B 1/041; A61B 1/043; A61B 1/00032; A61B 1/0684; A61B 1/00197; A61N 2005/0652; A61B 1/00158; A61B 1/00016; A61B 1/2736; A61N 5/062; A61N 2005/0609; A61B 1/00186

(57) ABSTRACT

The present invention provides a digestive tract capsule endoscopy integrated with photodynamic diagnosis and therapy; the digestive tract capsule endoscopy integrated with photodynamic diagnosis and therapy includes an imaging system, a control system, a battery and a communication system, and the imaging system includes a fluorescent camera, diagnosis LED light sources and therapy LED light sources. In the digestive tract capsule endoscopy integrated with photodynamic diagnosis and therapy provided by the present invention, it changes a conventional optical image system into a fluorescent image system, which requires no extra equipment and has strong adaptability; the present invention can achieve integrated diagnosis and therapy of human body's internal cavities, e.g., digestive tract and achieve the implementation of photodynamic diagnosis and therapy for one time; the digestive tract capsule endoscopy provided by the present invention can achieve diagnosis/therapy circulation, and can examine the therapeutic process of patients.

### TECHNICAL FIELD

The present invention belongs to the technical field of medical apparatus and instruments, and particularly relates to a digestive tract capsule endoscopy integrated with photodynamic diagnosis and therapy.

### BACKGROUND

Photodynamic Diagnosis (PDD) is a novel diagnostic technique, and the technique can selectively enrich cancer cells by a photosensitive drug and irradiate cancer tissues by exciting light to enable the enriched photosensitive drug to produce fluorescence, and then can analyze the intensity and distribution of the fluorescence, thus determining the position and situation of the cancer. For example, photosensitive drug ALA is a natural precursor of hemachrome way. Due to the increase of porphobilinogen deaminase activity and decrease of ferrochelatase activity, ALA exogenous application causes selective accumulation of hemachrome precursor protoporphyrin IX (PPIX) in cancer cells. PPIX is a kind of fluorescent substrate to give off red fluorescence under the stimulation of blue light (e.g., 385 nm). Researches show that ALA-PDD technique may significantly improve the detection sensitivity of peritoneal metastasis in stomach cancer. In addition, ALA-induced fluorescent guidance may used for the full resection of tumor and the improvement of progression free survival in malignant glioma operation. The greatest advantage of PDD is to distinguish tumor cells from normal cells by fluorescence, thus identifying a flat lesion and papilloma omitted in white light endoscopy, so as to take a therapeutic way in early stage of the tumor.

Currently, photodynamic diagnosis is conducted by capsule endoscopy, and capsule endoscopy is a kind of image system similar to capsule in its shape and size, and there is a complete set of digestive tract examination tools integrated with optical camera system, wireless communication system and power supply system in the capsule endoscopy. The capsule is swallowed by the subject to move in the gastrointestinal tract, thus shooting and examining the inner wall of gastrointestinal tract as well as sending images to external terminals. Physicians may locate and diagnose lesions according to the received images. Compared with conventional endoscope, capsule endoscopy has lower discomfortableness and higher patient's dependency. But the existing capsule endoscopy has single function and merely severs to shoot the white light image of the internal digestive tract, and its diagnosis accuracy depends on the physician's level, moreover, it is difficult to find out early cancer lesions by the technique, and it fails to implement PDT therapy directly. Such as, the existing disclosed patents are image-type capsule endoscopy: US 2005085696(A1) Medical apparatus, medical apparatus guide system, capsule type medical apparatus, and capsule type medical apparatus guide apparatus, CN101123694A imaging apparatus and endoscope apparatus using the same, CN202843565U medical magnetic capsule endoscope system.

Photodynamic therapy (PDT) is a kind of new method for treating tumor diseases by light sensitive effect, specifically, tumor sites are irradiated by specific-wavelength light, which may activate the photosensitive drug selectively gathered in tumor tissues to cause photochemical reaction, thus destroying the tumor. The photosensitive drug will transfer energy to the surrounding oxygen to generate singlet oxygen with strong activity. Oxidizing reaction performs between the singlet oxygen and nearby biomacromolecule to produce cytotoxicity, thus killing tumor cells. Compared with the conventional cancer therapy, PDT advantages are capable of achieving accurate, targeted and effective therapy, causing fewer side effects.

Photodynamic diagnosis PDD is combined with photodynamic therapy PDT to achieve precise treatment of tumor, and PDT is conducted with the indication of PDD photosensitizer (PS) fluorescence, thus bringing minimum damage to the surrounding normal tissues while treating the tumor fully. Meanwhile, at the end of therapy, PDD is conducted to determine the therapeutic effect, and secondary PDT therapy is conducted if necessary. Photodynamic diagnosis and therapy integrated technology is a development direction of accurate targeting therapy for tumor.

Specific to the photodynamic diagnosis and therapy integrated technology, an invention patent CN201810137077.1 discloses a diagnosis and therapy integrated photodynamic therapy system for cervical lesions, the system achieves the real-time detection of lesion shape, distribution and size as well as real-time and controllable photodynamic therapy by means of a coaxial light path; the system acquires lesion images by an imaging CCD probe, performs image edge detection by a computer and controls a LED profiling array to send out a light spot similar to the lesion, thus achieving accurate therapy. Such kind of system is large and has a complex structure, only suitable for body shallow cavities (oral cavity, nasal cavity, vagina, etc.), therefore, it fails to achieve photodynamic diagnosis and therapy integration to the tumor (e.g., stomach cancer) of internal cavities.

### SUMMARY

In view of this, an objective of the present invention is to provide a small digestive tract capsule endoscopy integrated with photodynamic diagnosis and therapy specific to gastrointestinal tumors.

The present invention provides a digestive tract capsule endoscopy integrated with photodynamic diagnosis and therapy, including an imaging system, a control system, a battery and a communication system, wherein, the imaging system comprises a fluorescent camera, diagnosis LED light sources and therapy LED light sources.

Preferably, the imaging system is located on one end of the digestive tract capsule endoscopy, the diagnosis LED light sources and the therapy LED light sources are radially spaced with the fluorescent camera as a center.

Preferably, any two adjacent diagnosis LED light source and the therapy LED light source are spaced by 40-50°.

Preferably, the central wavelength of the diagnosis LED light sources is 390-410 nm.

Preferably, the central wavelength of the therapy LED light sources is 620-640 nm.

Preferably, emitting angles of the diagnosis LED light sources and the therapy LED light sources are independently 55°-65°.

Preferably, secondary optical elements are arranged on surfaces of the diagnosis LED light sources and the therapy LED light sources.

Preferably, the secondary optical elements are lenses.

Preferably, the fluorescent camera is provided with a narrow-band filter, the central wavelength of the narrow-band filter is 397-403 nm, the bandwidth of the narrow-band filter is 30-100 nm; the minimum transmittance T of the narrow-band filter is >92%.

Preferably, the narrow-band filter is fixedly adhered on an outer surface of the fluorescent camera.

Beneficial effects of the present invention: a digestive tract capsule endoscopy integrated with photodynamic diagnosis and therapy provided by the present invention includes an imaging system, a control system, a battery and a communication system, and the imaging system includes a fluorescent camera, diagnosis LED light sources and therapy LED light sources. The digestive tract capsule endoscopy integrated with photodynamic diagnosis and therapy provided by the present invention is available by simply improving the imaging system of an existing magnetic-control endoscopic capsule system, that is, a conventional optical image system is replaced with a fluorescent image system, which requires no extra equipment and has strong adaptability; the present invention can achieve integrated diagnosis and therapy of human body's internal cavities, e.g., digestive tract and achieve the implementation of photodynamic diagnosis and therapy for one time; the digestive tract capsule endoscopy provided by the present invention frees patients from pain in treatment, thus greatly improving patients' treatment compliance; the digestive tract capsule endoscopy provided by the present invention can achieve diagnosis/therapy circulation, and can examine the therapeutic process of patients.

### BRIEF DESCRIPTION OF THE DRAWINGS

FIG. 1 is a schematic diagram of a digestive tract capsule endoscopy integrated with photodynamic diagnosis and therapy;

FIG. 2 is a work flow diagram of the digestive tract capsule endoscopy integrated with photodynamic diagnosis and therapy;

FIG. 3 is a schematic diagram showing the diagnosis and therapy of the digestive tract capsule endoscopy integrated with photodynamic diagnosis and therapy.

### DETAILED DESCRIPTION

The present invention provides a digestive tract capsule endoscopy integrated with photodynamic diagnosis and therapy, including an imaging system, a control system, a battery and a communication system, where the imaging system includes a fluorescent camera, diagnosis LED light sources and therapy LED light sources.

In the present invention, the digestive tract capsule endoscopy integrated with photodynamic diagnosis and therapy is obtained by improving the imaging system of the existing magnetic-control endoscopic capsule system; in the present invention, the imaging system of the magnetic-control endoscopic capsule system includes a fluorescent camera, diagnosis LED light sources and therapy LED light sources. In the present invention, the imaging system is preferably located on one end of the digestive tract capsule endoscopy. In the present invention, the imaging system preferably includes a fluorescent camera, several diagnosis LED light sources and several therapy LED light sources; the diagnosis LED light sources and the therapy LED light sources are radially spaced preferably with the fluorescent camera as a center. In the present invention, the number of the diagnosis LED light sources and the therapy LED light sources is independently and preferably 3-5, and more preferably 4; any two adjacent diagnosis LED light source and the therapy LED light source are spaced by preferably 40-50°, and more preferably 45°.

In the present invention, there is no special limitation to the central wavelength of the diagnosis LED light sources and the therapy LED light sources, any central wavelength of a LED luminescent source capable of achieving diagnosis and therapy is available, and in the present invention, the central wavelength of the diagnosis LED light sources and the therapy LED light sources may be matched and upgraded according to the optical property of commercially available photosensitizers. In detailed implementation process of the present invention, specific to the existing photosensitizers, the central wavelength of the diagnosis LED light sources is preferably 390-410 nm, and more preferably 400 nm; the central wavelength of the diagnosis LED light sources is preferably 620-640 nm, and more preferably 630 nm.

In the present invention, emitting angles of the diagnosis LED light sources and the therapy LED light sources are independently and preferably 55°-65°, and more preferably 60°; in the present invention, emitting angles of the diagnosis LED light sources and the therapy LED light sources are preferably achieved by setting secondary optical element on the surface thereof. In the present invention, the secondary optical element is preferably a lens; there is no special limitation to the source and specification of the lens in the present invention, in detailed implementation process of the present invention, the lens is preferably customized from the manufacturer of the LED luminescent sources, and the manufacturer machines the lens with corresponding numerical value and texture on the surface of LED chips directly during LED packaging, so that the emitting angles of the LED luminescent sources are 55°-65°.

In the present invention, the fluorescent camera is preferably provided with a narrow-band filter, and the narrow-band filter is preferably fixedly adhered on an outer surface of the fluorescent camera. In the present invention, the central wavelength of the narrow-band filter is preferably 397-403 nm, the bandwidth of the narrow-band filter is preferably 30-100 nm; the minimum transmittance T of the narrow-band filter is preferably >92%. In the present invention, the narrow-band filter serves to filter out the exciting light of photodynamic diagnosis PDD, and only allows fluorescence generated by stimulation to pass through and to be captured by a camera.

In the present invention, there is no special limitation to the control system, battery and the communication system of the digestive tract capsule endoscopy integrated with photodynamic diagnosis and therapy as long as a control system, a battery and a communication system of an existing digestive tract capsule endoscopy are taken available. In detailed implementation process of the present invention, the existing digestive tract capsule endoscopy is a “magnetic-control capsule gastroscope system robot” product produced by Anhan Optoelectronic Technology (Wuhan) Co., Ltd.; the product and product structure are specifically referring to a Chinese patent CN201310275948.3. The solution of the present invention mainly aims at an illuminating system and an image capture camera of the capsule endoscopy, and does not involve other parts of the digestive tract capsule endoscopy, such as a capsule shell, an optoelectronic switch device, an image information processing circuit, a radio frequency transmission device, a power unit, a magnetic field sensor, a small magnet; the above parts are not limited by the present invention as long as the structure or component capable of achieving related functions in the field is taken available.

The technical solution provided by the present invention will be described in detail in connection with the following embodiments, but they should not be construed as a limitation to the claimed scope of the present invention.

### Embodiment 1

Digestive Tract Capsule Endoscopy Integrated with Photodynamic Diagnosis and Therapy

The capsule endoscopy includes an imaging system, a control system, a battery and a communication system, and the imaging system includes a fluorescent camera, diagnosis LED light sources and therapy LED light sources.

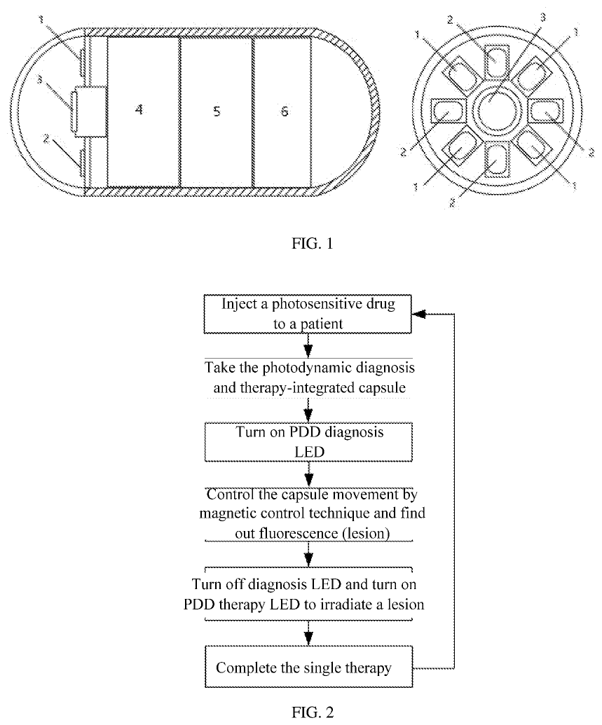

The concrete structure is shown in the left of FIG. 1, where 1 denotes diagnosis LED light sources, 2 denotes therapy LED light sources, 3 denotes fluorescent camera, 4 denotes control system, 5 denotes battery and 6 denotes communication system. The positional relation of the fluorescent camera, diagnosis LED light sources and therapy LED light sources is shown in the right of FIG. 1, the diagnosis LED light sources and the therapy LED light sources are radially spaced with the fluorescent camera as a center; the number of diagnosis LED light sources and therapy LED light sources is respectively 4, and the interval angles are 45°.

A fluorescent camera is arranged in a front end of the endoscope, a 400 nm-central wavelength narrow-band filter is fixedly adhered on an outer surface of the camera, bandwidth is 30 nm; minimum transmittance: T>92%; the narrow-band filter may filter out PDD exciting light, and only allow fluorescence generated by stimulation to pass through and to be captured by a camera.

The structure of other parts, namely, control system, battery and communication system are referring to the records of the Chinese patent CN201310275948.3.

### Embodiment 2

The digestive tract capsule endoscopy integrated with photodynamic diagnosis and therapy in Embodiment 1 is applied in photodynamic diagnosis and therapy.

The integrated diagnosis and therapy process of stomach cancer is set as an example, and the workflow is shown in FIG. 2:

(1) a suspected stomach cancer patient did not take food at least 8 h before receiving therapy;

(2) a photosensitive drug (e.g., HPD, 5 mg/kg body weight) was injected to make the photosensitive drug enriched in tumor tissues;

(3) the stomach cancer patient fetched and took the therapy capsule with 1.5-2 L water;

(4) 1.5-2 L water made stomach full, so that diagnosis and therapy were completed smoothly;

(5) a physician turned on PDD diagnosis LED and probed the stomach by a magnetic control system;

(6) the capsule was suspended after finding a fluorescence enhancement region (tumor tissues);

(7) the physician turned off diagnosis LED and turned on PDT therapy LED to irradiate lesions (power density was 20 mW/cm2 and irradiation time was 20-50 min), photochemical reaction performed between the therapy light and the enriched photosensitive drug to generate singlet oxygen toxicant to cells, thus killing tumor cells purposefully;

(8) at the end of therapy, the internal electric source of the capsule exhausted, and the capsule would be discharged through small intestine, large intestine and anus within 1-2 d;

(9) after finishing the primary therapy, the physician may select a proper day to pre-probe and treat lesions by the diagnosis and therapy integrated capsule once again, thus achieving healing, and tumor cells were not found by tissue biopsy.

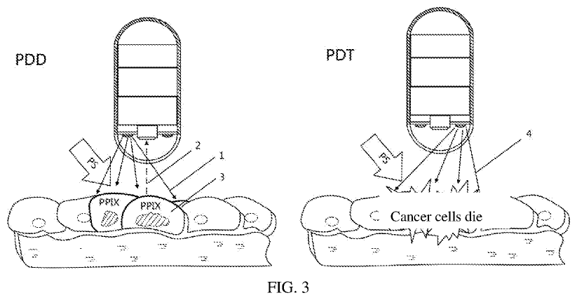

The schematic diagram of the diagnosis and therapy of the digestive tract capsule endoscopy integrated with photodynamic diagnosis and therapy is shown in FIG. 3, of which 1 denotes PDD diagnosis LED light sources; 2 denotes fluorescence emitted by tumor tissues; 3 denotes tumor tissues; 4 denotes PDT therapy LED light sources.

The foregoing descriptions are only preferred implementation manners of the present invention. It should be noted that for a person of ordinary skill in the art, several improvements and modifications may further be made without departing from the principle of the present invention. These improvements and modifications should also be deemed as falling within the protection scope of the present invention.

1. A digestive tract capsule endoscopy integrated with photodynamic diagnosis and therapy, comprising an imaging system, a control system, a battery and a communication system, wherein, the imaging system comprises a fluorescent camera, diagnosis LED light sources and therapy LED light sources.

2. The digestive tract capsule endoscopy according to claim 1, wherein the imaging system is located on one end of the digestive tract capsule endoscopy, the diagnosis LED light sources and the therapy LED light sources are radially spaced with the fluorescent camera as a center.

3. The digestive tract capsule endoscopy according to claim 2, wherein any two adjacent diagnosis LED light source and the therapy LED light source are spaced by 40-50°.

4. The digestive tract capsule endoscopy according to claim 1, wherein the central wavelength of the diagnosis LED light sources is 390-410 nm.

5. The digestive tract capsule endoscopy according to claim 1, wherein the central wavelength of the therapy LED light sources is 620-640 nm.

6. The digestive tract capsule endoscopy according to claim 1, wherein emitting angles of the diagnosis LED light sources and the therapy LED light sources are independently 55°-65°.

7. The digestive tract capsule endoscopy according to claim 6, wherein secondary optical elements are arranged on surfaces of the diagnosis LED light sources and the therapy LED light sources.

8. The digestive tract capsule endoscopy according to claim 7, wherein the secondary optical elements are lenses.

9. The digestive tract capsule endoscopy according to claim 1, wherein the fluorescent camera is provided with a narrow-band filter, the central wavelength of the narrow-band filter is 397-403 nm, the bandwidth of the narrow-band filter is 30-100 nm; the minimum transmittance T of the narrow-band filter is >92%.

10. The digestive tract capsule endoscopy according to claim 9, wherein the narrow-band filter is fixedly adhered on an outer surface of the fluorescent camera.

11. The digestive tract capsule endoscopy according to claim 2, wherein the central wavelength of the diagnosis LED light sources is 390-410 nm.

12. The digestive tract capsule endoscopy according to claim 3, wherein the central wavelength of the diagnosis LED light sources is 390-410 nm.

13. The digestive tract capsule endoscopy according to claim 2, wherein the central wavelength of the therapy LED light sources is 620-640 nm.

14. The digestive tract capsule endoscopy according to claim 3, wherein the central wavelength of the therapy LED light sources is 620-640 nm.

15. The digestive tract capsule endoscopy according to claim 2, wherein emitting angles of the diagnosis LED light sources and the therapy LED light sources are independently 55°-65°.

16. The digestive tract capsule endoscopy according to claim 3, wherein emitting angles of the diagnosis LED light sources and the therapy LED light sources are independently 55°-65°.

17. The digestive tract capsule endoscopy according to claim 15, wherein secondary optical elements are arranged on surfaces of the diagnosis LED light sources and the therapy LED light sources.

18. The digestive tract capsule endoscopy according to claim 16, wherein secondary optical elements are arranged on surfaces of the diagnosis LED light sources and the therapy LED light sources.

19. The digestive tract capsule endoscopy according to claim 17, wherein the secondary optical elements are lenses.

20. The digestive tract capsule endoscopy according to claim 2, wherein the fluorescent camera is provided with a narrow-band filter, the central wavelength of the narrow-band filter is 397-403 nm, the bandwidth of the narrow-band filter is 30-100 nm; the minimum transmittance T of the narrow-band filter is >92%.
